(19) United States
(12) Patent Application Publication
(10) Pub. No.: US 2017/0100506 A1
(43) Pub. Date: Apr. 13, 2017

(54) PROTEASE RESISTANT GROWTH FACTOR FORMULATIONS FOR CHRONIC WOUND HEALING

(71) Applicants: Yuan Yuan, Brandon, FL (US); Piyush Koria, Tampa, FL (US)

(72) Inventors: Yuan Yuan, Brandon, FL (US); Piyush Koria, Tampa, FL (US)

(73) Assignee: University of South Florida, Tampa, FL (US)

(21) Appl. No.: 15/290,742

(22) Filed: Oct. 11, 2016

Related U.S. Application Data
(60) Provisional application No. 62/238,848, filed on Oct. 8, 2015.

(51) Int. Cl. A61L 26/00

(52) U.S. Cl. CPC A61L 26/0047; A61L 26/0052; A61L 2300/252; A61L 2300/45

(57) ABSTRACT

A formulation and method of treating chronic wounds is presented. The formulation uses two different fusion peptides, one of which incorporates an elastase resistant peptide, to preserve the bioactivity of different functional peptides and growth factors in chronic wounds.

### CROSS REFERENCE TO RELATED APPLICATIONS

This application is a nonprovisional of and claims priority to U.S. Provisional Application No. 62/238,848, entitled “Protease Resistant Growth Factor Formulations for Chronic Wound Healing”, filed Oct. 8, 2015, the contents of which are herein incorporated by reference.

### FIELD OF INVENTION

This invention relates to a formulation and method for chronic wound treatment.

Specifically, a formulation and method of using an elastase inhibition fusion peptide during growth factor therapy is presented.

### BACKGROUND OF THE INVENTION

Wound healing is a complex process that generally consists of three main phases: inflammation, proliferation and remodeling. The inflammatory phase begins at the time of injury and lasts 2-4 days. The phase begins with hemostasis and formation of the platelet plug. Collagen that is exposed during wound formation activates the clotting cascade. After injury to tissue occurs, the damaged cell membranes release thromboxane A2 and prostaglandin 2α, both of which are potent vasoconstrictors, to help limit hemorrhage. After a short time, capillary vasodilation occurs secondary to local histamine release and the cells of inflammation are able to migrate to the wound bed. Platelets release platelet-derived growth factor (PDGF) and transforming growth factor beta (TGF-β) from their alpha granules to attract neutrophils and macrophages while neutrophils scavenge for bacteria and foreign debris. Macrophages are the most important mediators of wound healing and continue to emit growth factors to attract fibroblasts and usher in the next phase of wound healing.

The proliferative phase begins on approximately day 3, overlapping with the inflammatory phase. Fibroblasts are responsible for initiating angiogenesis, epithelialization, and collagen formation and peak approximately day 7 from injury. Epithelialization occurs from the basement membrane if the basement membrane remains intact (e.g., first-degree burn). If the basement membrane is not intact, then epithelialization occurs from the wound edges. Fibroblasts produce mainly type III collagen during this phase. Granulation tissue, formed in this phase, is particularly important in wound healing by secondary intention. When collagen synthesis and breakdown become equal, the next phase of wound healing has begun.

In the remodeling phase, increased collagen production and breakdown continue for 6 months to 1 year after injury. The initial type III collagen is replaced by type I collagen until a type I:type II ratio of 4:1 is reached, which is equal to normal skin. Fibroblasts differentiate into myofibroblasts which causes tissue contraction during this phase of wound healing. Collagen reorganizes along lines of tension and crosslinks to give added strength. Strength eventually approaches 80% of the strength of uninjured tissue. Vascularity decreases, producing a less hyperemic and more cosmetically appealing wound as this phase progresses.

The timetable for wound healing can be variable with chronic wounds often stalling in the inflammatory phase due to poor perfusion, poor nutrition, or other factors which cause excessive buildup of exudates in the wound base. Chronic wounds tend to remain unhealed unless active and aggressive methods are undertaken. Chronic wounds generally exhibit the following characteristics: low mitogenic activity; increased number of inflammatory cytokines; increased levels of proteases; and senescent cells present in the wound bed.

Growth factor therapy has been proved as a promising treatment for chronic wounds. Growth factor therapy, as used with respect to wound healing, refers to the use of substances secreted by the body that function to stimulate the growth and proliferation of the cells involved in wound healing and inflammation thus resulting in faster wound healing. Examples of some growth factors that may be used in growth factor therapy of chronic wound healing include: epidermal growth factor (EGF); keratinocyte growth factor (KGF); transforming growth factors (TGF); vascular endothelial growth factor (VEGF); platelet-derived growth factor (PDGF); fibroblast growth factor (FGF); interleukins (IL); and colony-stimulating factors (CSF).

EGF is secreted by platelets and macrophages and stimulates the proliferation of fibroblasts. EGF also stimulates fibroblasts to secrete collagenase to degrade the matrix during the remodeling phase and may reduce healing time if applied topically.

KGF promotes the migration, differentiation and proliferation of keratinocytes. KGF is secreted by fibroblasts and plays a prominent role in wound healing by enhancing re-epithelialization of the wound.

TGFs can be secreted by platelets, macrophages, lymphocytes and hepatocytes. TGF-α stimulates growth and migration of keratinocytes and fibroblasts to an affected area. TGF-β1 and TGF-β2 promote angiogenesis; upregulate collagen production and inhibit degradation; and promote chemoattraction of inflammatory cells. TGF-β, which is an antagonist to TGF-β1 and TGF-β2, has been discovered in high levels in fetal scarless wound healing and has promoted scarless healing in adults experimentally when TGF-β1 and TGF-β2 are suppressed.

VEGF is secreted by endothelial cells and promotes angiogenesis during tissue hypoxia.

PDGF is secreted by platelets, macrophages and endothelial cells. PDGF attracts fibroblasts and macrophages to the affected area and promotes collagen and proteoglycan synthesis.

FGF is secreted by macrophages, mast cells and T-lymphocytes. FGF promotes angiogenesis, granulation and epithelialization via endothelial cell, fibroblast and keratinocyte migration, respectively.

Interleukins are secreted by macrophages, keratinocytes, endothelial cells, lymphocytes, fibroblasts, osteoblasts, basophils and mast cells. IL-1 is a proinflammatory chemotactic for neutrophils, fibroblasts, and keratinocytes and also activates neutrophils. IL-4 activates fibroblast differentiation and induces collagen and proteoglycan synthesis. IL-8 is a chemotactic for neutrophils and fibroblasts.

CSFs are secreted by stromal cells, fibroblasts, endothelial cells and lymphocytes. Granulocyte colony stimulating factor (G-CSF) stimulates granulocyte proliferation. Granulocyte macrophage colony stimulating factor (GM-CSF) stimulates granulocyte and macrophage proliferation.

Growth factors for wound healing can be applied topically or injected. In some cases, growth factors can be incorporated into wound dressings or commercially available skin grafts.

As stated above, chronic wounds tend to have increased levels of proteases. Increased levels of proteases lead to the degradation of proteins such as growth factors which results in chronic wounds staying the inflammatory phase which prevents the wound from progressing to the proliferative phase of wound healing. Normally in wound healing, there is an initial rapid increase in protease levels which peaks at about day three and reduces by about day 5. In chronic wounds, the protease levels not only reach higher levels, the high levels persist much longer. In wound healing, the major proteases are matrix metalloproteinases (MMPs) and serine proteases such as elastase. Elastase is a serine protease found in the highest concentrations in the elastic fibers of connective tissues which is capable of digesting and degrading a number of proteins including elastin. The primary source of elastase is neutrophils but it may also be produced by bacteria. It is one of the primary destructors of peptide growth factors and also has negative effects on fibroblasts. (Wound Healing: Evidence-Based Management, edited by Joseph McCulloch and Luther Kloth, F.A. Davis Co., 2010, 4^{th} ed., pp. 189)

Elastin-like peptides (ELPs) are biopolymers that consist of pentameric repeats of (Val-Pro-Gly-X-Gly)n, where X is a “guest” residue that can be anything other than Pro. ELPs reversibly transition from soluble to insoluble based on an inverse phase transition temperature (Tₜ). ELPs are soluble below their transition temperature and are susceptible to degradation by elastase and collagenase. Changing the ELP length, n, and the guest residue, X, allows for accurate and reproducible control of the transition temperature. ELPs with different transition temperatures can be fused together to form block copolymers that assemble micelle nanoparticles. Biodegradation of ELP nanoparticles is more complicated since they contain both soluble and insoluble ELP blocks. With regard to the micellar nanoparticles, it was found that collagenase activity is slightly moderated by micelle formation, however elastase activity is the same regardless of incubation temperature. (Shah, Mihir et al., Biodegradation of elastin-like polypeptide nanoparticles, Protein Science (2012), 21(6):743-750)

Numerous growth factors have been tested in animal model and shown a positive effect on wound healing, however due to high protease levels in chronic wounds, repeated administration is required with daily administration being needed in many instances. Further, the stability of therapeutic drugs and growth factors in chronic wound treatment is a significant challenge. Accordingly, what is needed is a novel formulation for chronic wound treatment that reduces the frequency of administration while preserving the bioactivity of growth factors.

### SUMMARY OF INVENTION

The inventors propose a novel formulation that can preserve the bioactivity of different growth factors and functional peptides in chronic wounds. Through inhibition of elastase, the formulation can reduce the dosage that needed for the treatment. Moreover, the elastin-like peptide (ELP) backbone of the fusion protein allows formation of nanoparticles with different growth factor combinations for chronic wound treatment, which has an impact on multiple regeneration processes.

In an exemplary embodiment, the composition described in this invention incorporates a variant of PMP-D2, R29L, an elastase inhibition peptide, to preserve the bioactivity of the cargo protein from protease digestion which occurs in chronic wound treatments. The fusion protein retains the transition property of elastin-like peptide (ELP) which allows the expression and purification of large quantities of the protein rapidly through ITC. Retaining the bioactivity of the cargo protein also helps reduce the dosage and number of treatments needed by the patient. Moreover, the ELP backbone of the fusion protein allows formation of nanoparticles with different growth factors combination for chronic wound treatment which is critical since wound healing involves different regeneration processes such as cell proliferation, migration and angiogenesis. The fusion proteins described herein increase the efficiency of growth factor therapy by retaining their bioactivity in the chronic wound area.

In an embodiment of the invention, a nanoparticle composition for treating chronic wounds is presented comprising: a first fusion peptide comprising an elastase resistant peptide bound to a polypeptide backbone; a second fusion peptide comprising a cargo peptide bound to a polypeptide backbone; and a pharmaceutically acceptable carrier. The first fusion peptide and the second fusion peptide self-assemble into a heterogeneous nanoparticle in response to adjustment of transition temperature of the polypeptide backbone. The polypeptide backbone can be an elastin like peptide (ELP). The elastase resistant peptide can be a PMP-D2 variant, such as R29L or R29L/K30M. The cargo peptide can be a growth factor or a functional peptide. The growth factor can be selected from the group consisting of epidermal growth factor (EGF); keratinocyte growth factor (KGF); transforming growth factors (TGF); vascular endothelial growth factor (VEGF) including BMP-2; platelet-derived growth factor (PDGF); fibroblast growth factor (FGF); interleukins (IL); colony-stimulating factors (CSF) and combinations thereof. The functional peptide can be cathelicidin (LL37).

In a further embodiment, a method of healing chronic wounds is presented in which a nanoparticle composition comprised of a first fusion peptide comprising an elastase resistant peptide bound to an elastin like peptide (ELP); a second fusion peptide comprising a cargo peptide bound to an elastin like peptide (ELP); and a pharmaceutically acceptable carrier. The first fusion peptide and the second fusion peptide self-assemble into a heterogeneous nanoparticle in response to adjustment of transition temperature of the ELPs. The elastase resistant peptide can be a PMP-D2 variant, such as R29L or R29L/K30M. The cargo peptide can be a growth factor or a functional peptide. The growth factor can be selected from the group consisting of epidermal growth factor (EGF); keratinocyte growth factor (KGF); transforming growth factors (TGF); vascular endothelial growth factor (VEGF) including BMP-2; platelet-derived growth factor (PDGF); fibroblast growth factor (FGF); interleukins (IL); colony-stimulating factors (CSF) and combinations thereof. The functional peptide can be cathelicidin (LL37).

In some embodiments, the elastase resistant peptide in the first fusion peptide can be substituted with an MMP resistant peptide.

### BRIEF DESCRIPTION OF THE DRAWINGS

For a fuller understanding of the invention, reference should be made to the following detailed description, taken in connection with the accompanying drawings, in which:

FIG. 1A is a graph depicting protease activity levels associated with the healing status of chronic wounds.

FIG. 1B is an image depicting a western blot that illustrates that Platelet Derived Growth Factor (PDGF) is degraded by elastase in 2 hrs.

FIG. 2A is an image depicting PMP-D2 which is composed of 35 residues, which are cross-linked by three disulfide bonds.

FIG. 2B is an image depicting elastin like peptides (ELPs) which are biodegradable, non-immunogenic protein-based polymers composed of tandemly repeated blocks of (Val-Pro-Gly-X-Gly)n where X can be any residue but Pro. As shown in the image, when the ELPs are present below their transition temperature (Tt), they are soluble in aqueous solutions while when they are present in solution above the Tt, they are insoluble and are capable of self-assembly into nanoparticles.

FIG. 3 is an image depicting a heterogeneous nanoparticle, wherein fusion peptides comprising of PMP-D2 (yellow) or cargo (red) and ELPs self-assemble into a heterogeneous nanoparticle.

FIG. 4 is a graph depicting the results of an elastase activity assay in which ELP and PMP alone are compared to a control.

FIG. 5A is a western blot depicting that PMP-D2 ELP fusion retains growth factors bioactivity in accordance with an exemplary embodiment of the present invention.

FIG. 5B is a graph depicting that PMP-D2 ELP fusion retains growth factors bioactivity in accordance with an exemplary embodiment of the present invention.

FIG. 6A is a series of images depicting ELP and PDGF both show promotion on re-epithelialization when compared to the control in the case of a diabetic mouse (B6.BKS-Leprdb) model.

FIG. 6B is a series of images tracking the degradation process of ELP in vivo in which ELP conjugated with biotin to treat diabetic mice is used.

FIG. 7A is a series of images illustrating the delay in the wound closure of the experimental mice in the presence of human leukocyte elastase (HLE).

FIG. 7B is a graph depicting wound closure results in the presence of human leukocyte elastase (HLE) in the case of the experimental mice.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

### DEFINITIONS

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. As used herein, “about” refers to ±10%.

As used in the specification and claims, the singular form “a”, “an” and “the” include plural references unless the context clearly dictates otherwise. For example, the term “a nanoparticle” includes a plurality of nanoparticles, including mixtures thereof.

“Patient” is used to describe an animal, preferably a human, to whom treatment is administered, including prophylactic treatment with the compositions of the present invention. “Patient” and “subject” are used interchangeably herein.

The “therapeutically effective amount” for purposes herein is thus determined by such considerations as are known in the art. A therapeutically effective amount of the formulations described herein is that amount necessary to provide a therapeutically effective result in vivo. The amount of nanoparticle composition containing fusion proteins must be effective to achieve a response, including but not limited to improvement or elimination of symptoms associated with inflammatory disorders, such as chronic wounds.

“Administration” or “administering” is used to describe the process in which the nanoparticle composition of fusion proteins of the present invention are delivered to a patient. The composition may be administered topically, such as a transdermal patch, a spot-on treatment or an ointment.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. In some embodiments, the pharmaceutically acceptable carrier is a gel, ointment, hydrogel, cream, aerosol, or powder. In some embodiments, the pharmaceutically acceptable carrier is a gel, such as a fibrin gel. Examples of other pharmaceutically acceptable carriers include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention.

“Treatment” or “treating” as used herein refers to any of: the alleviation, amelioration, elimination and/or stabilization of a symptom, as well as delay in progression of a symptom of a particular disorder. For example, “treatment” of chronic wounds may include any one or more of the following: amelioration and/or elimination of one or more symptoms associated with chronic wounds, reduction of one or more symptoms of chronic wounds, stabilization of symptoms of chronic wounds, and delay in progression of one or more symptoms of chronic wound.

“Elastin-like peptide (ELP)” as used herein refers to biodegradable, non-immunogenic protein-based polymers composed of tandemly repeated blocks of (Val-Pro-Gly-X-Gly)n where X can be any residue but Pro and n is the number of repeated blocks (length of the ELP).

“Elastase resistant peptide” as used herein refers to a peptide which is shown to inhibit elastase in chronic wounds. Examples of elastase resistant peptides include, but are not limited to, PMP-D2 variants R29L and R29LUK30M.

“Matrix metalloproteinase (MMP) resistant peptide” as used herein refers to a peptide which is shown to inhibit MMPs in chronic wounds. Several MMP resistant peptides are contemplated for use in the instant invention.

“Growth factor” as used herein refers to substances secreted by the body that function to stimulate the growth and proliferation of the cells involved in wound healing and inflammation thus resulting in faster wound healing. Examples of growth factors that may be used in growth factor therapy of chronic wound healing include, but are not limited to: epidermal growth factor (EGF); keratinocyte growth factor (KGF); transforming growth factors (TGF); vascular endothelial growth factor (VEGF) including BMP-2; platelet-derived growth factor (PDGF); fibroblast growth factor (FGF); interleukins (IL); colony-stimulating factors (CSF) and combinations thereof.

“Chronic wound” as used herein refers to a wound which lingers in the inflammatory phase of wound healing without advancing to the proliferative phase and is thus unable to heal. Wounds such as diabetic, venous or decubitus ulcers are considered to be chronic wounds. The term “wound” as used herein refers to injuries to living tissue. In some embodiments, “wound” refers to injuries to the skin.

“Fusion peptide” or “fusion protein” as used herein refers to a peptide in which a bioactive molecule is attached to a polypeptide backbone. In some embodiments, two fusion peptides are used with the first fusion peptide comprising the bioactive molecule being an elastase-resistant peptide and the polypeptide backbone being comprised of elastin-like peptides (ELP) and the second fusion peptide comprising the bioactive molecule being a growth factor or functional peptide and the polypeptide backbone being comprised of ELPs. The fusion proteins described herein are capable of preserving the bioactivity of different functional peptides and growth factors and can self-assemble into nanoparticles dependent on transition temperature.

“Bioactive molecule” as used herein refers to a peptide or growth factor which is capable of exerting a beneficial biological effect on a wound of a patient. Bioactive molecules include, but are not limited to, functional peptides, MMP resistant peptides, elastase resistant peptides, and growth factors.

“Functional peptide” as used herein refers to a biological protein or peptide which exerts a beneficial biological effect on wound healing. In some embodiments, the peptide is an antimicrobial peptide which acts to have a beneficial biological effect on an infection. Examples of an antimicrobial functional peptide for use in the instant invention include, but are not limited to, SR-0379 and cathelicidin (LL-37), which is a small cationic antimicrobial peptide that plays a critical role against invasive bacterial infection. While examples of antimicrobial functional peptides are given, the invention contemplates all antimicrobial peptides. In some embodiments, growth factors are also considered functional peptides.

A “beneficial biological effect” as used herein refers to exhibition of an effect that is associated with wound healing. Examples of beneficial biological effects include, but are not limited to, re-epithelialization, granulation, angiogenesis, upregulation of collagen production, etc.

“Cargo peptide” as used herein refers to a functional peptide or growth factor that is part of the second fusion protein. Self-assembly regulated by the transition temperature of the ELPs allows the first fusion protein and the second fusion protein to form a nanoparticle used for the drug delivery system in the present invention.

The invention disclosed describes development of a formulation or composition that preserves the bioactivity of different functional peptides and growth factors in chronic wounds by incorporation of an elastase resistant peptide. The terms “formulation” and “composition” are used interchangeably herein.

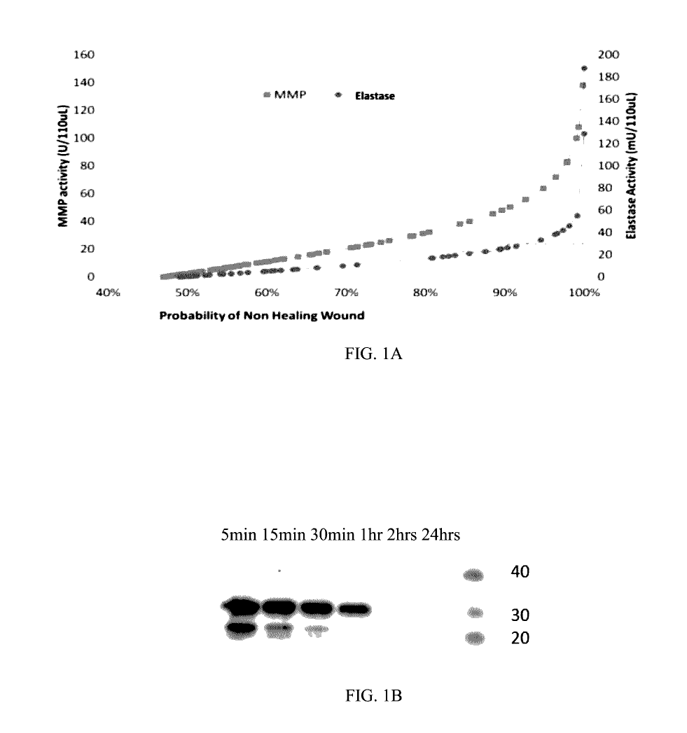

Elastase levels found in wound areas is closely related to the probability of chronic wounds. FIG. 1A illustrates protease activity levels associated with the healing status of chronic wounds. As depicted in the image, high MMP and elastase levels are associated with an increased probability of the wound not healing. The increased protease levels in chronic wounds contribute to degradation of growth factors which also hinders healing of the wound. FIG. 1B is a western blot illustrating that platelet derived growth factor (PDGF) is degraded by elastase in 2 hrs. A high elastase level is not only an important marker but also undermines the efficiency of growth factor therapy for chronic wounds.

Serine protease inhibitors, specifically elastase inhibitors, are of particular interest for use in treating chronic wounds. An exemplary serine protease inhibitor is the PMP-D2 variant. PMP-D2 is composed of 35 residues cross-linked by three disulfide bonds. The core region adopts a very similar, compact, globular fold, which consists of three strands (β1, β2 and β3) arranged in an anti-parallel 3 sheet that demarcates a cavity and an amino-terminal segment, oriented almost perpendicular to the P sheet. Inside the cavity, hydrophobic residues are clustered with an aromatic ring in the center of the hydrophobic core. The specificity of serine protease inhibitors has been shown to be specifically, but not exclusively, dependent on the nature of the P1 residue. Elastase inhibitors have been shown to prefer small hydrophobic residues, such as Val and Ala, at the P1 position. (Simonet, G. et al., Structural and functional properties of a novel serine protease inhibiting peptide family in arthropods, 2001, Comparative Biochemistry and Physiology, Part B 132:247-255) Variant R29L has shown a strong inhibition activity against several proteases. Additionally, several elastase inhibitors have been reported to have a Met residue at P′1, such as mucous proteinase inhibitor and Ascaris trypsin/elastase inhibitor. A double variant of PMP-D2, R29L/K30M (with Met as P′1), has also been shown to be a strong elastase inhibitor. (Kellenberger, C. et al., Serine protease inhibition by insect peptides containing a cysteine knot and a triple-stranded 13-sheet, 1005, The Journal of Biological Chemistry, 270(43):25514-25519)

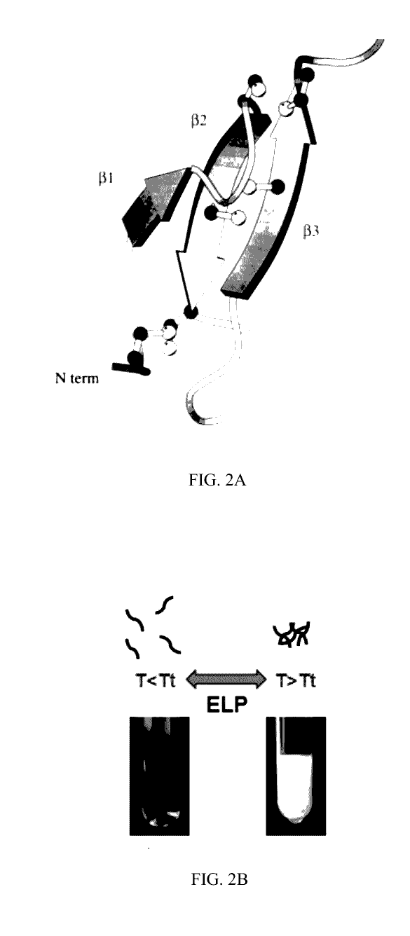

Elastin like peptides (ELPs) are biodegradable, non-immunogenic protein-based polymers composed of tandemly repeated blocks of (Val-Pro-Gly-X-Gly)n where X can be any residue but Pro and n is the number of repeated blocks (length of the ELP). (FIG. 2B). The sequence motif is derived from the hydrophobic domain of tropoelastin, a soluble precursor form of elastin. As stated above, ELPs can reversibly transition from soluble to insoluble based on an inverse phase transition temperature (Tₜ). ELPs are soluble below their transition temperature and are susceptible to degradation by elastase and collagenase. Changing the ELP length, n, and the guest residue, X, allows for accurate and reproducible control of the transition temperature. ELPs with different transition temperatures can be fused together to form block copolymers that assemble micelle nanoparticles. By exploiting the phase transitioning property of ELPs, a temperature responsive drug delivery system is created.

Fusion proteins in which growth factors are fused to a polypeptide backbone have had success in treating chronic wounds. For example, in the study by Koria et al, herein incorporated by reference in its entirety into this disclosure, it was found that a fusion protein comprising KGF and ELPs was beneficial for use in chronic wound treatment, The KGF-ELP fusion protein showed enhanced re-epithelialization and granulation as compared to controls of free KGF and free ELPs. The KGF-ELP fusion protein also showed enhanced results as compared to just blending KGF with ELP particles. (Koria, P. et al., Self-assembling elastin-like peptides growth factor chimeric nanoparticles for the treatment of chronic wounds, 2011, PNAS, 108(3): 1034-1039).

In accordance with an embodiment of the present invention, the building blocks of the inventive formulation comprise two sets of fusion proteins which self-assemble into heterogeneous nanoparticles. The first fusion peptide is comprised of an elastase resistant peptide, such as the PMP-D2 variant R29L, fused to a polypeptide backbone of elastin-like polypeptides (ELPs). The second fusion peptide is comprised of a bioactive protein or growth factor such as keratinocyte growth factor (KGF), bone morphogenetic protein 2 (BMP2), or functional peptide like cathelicidin (LL37) fused to a polypeptide backbone of ELPs. The fusion proteins retain the biological activities of the fused moieties as well as the characteristic phase transitioning properties of ELPs. These proteins are encoded in plasmid, which are then expressed in bacteria and purified by exploiting the phase transitioning behavior of ELPs. In some embodiments, the first fusion peptide is comprised of an MMP resistant peptide, instead of an elastase resistant peptide, fused to ELPs.

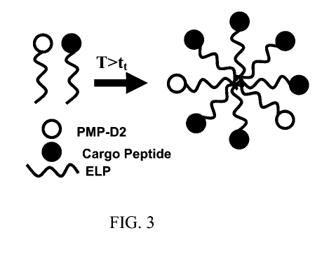

FIG. 3 is an illustration of a heterogeneous nanoparticle, wherein fusion peptides, comprising of PMP-D2 (yellow) or cargo (red), and ELPs self-assemble into a heterogeneous nanoparticle. As illustrated in FIG. 3, these proteins self-assemble into heterogeneous nanoparticles at their transition temperatures. Compared to free delivery of the cargo protein or peptide, these nanoparticles retain their bioactivities longer in a high protease environment.

In an experimental embodiment, genes encoding PMP-D2 variant R29L were excised and cloned in frame with the gene encoding the ELP cassette V40C2; V=VPGVG, (C=VPGVGVPGVGVPGCGVPGVGVPGVG) at the N-terminus of the ELP cassette. This yielded a gene encoding the building blocks of the form PMPD2-ELP. This gene was then cloned into an expression plasmid (pET25b+) and was expressed in bacteria (BLR cells). The expression of the fusion proteins was verified using western blots.

For purification, overnight-grown bacteria were lysed using two twelve-minute cycles in a sonicator. Each cycle consisted of alternating on/off minutes for the sonicator. The PMPD2-ELP was transitioned using salt and incubating the solution at 40′C. The PMPD2-ELP was pelleted by a hot spin with the supernatant being discarded. The PMPD2-ELP in the pellet was solubilized at 4′C using a buffer containing DTT. The solution was then centrifuged at 4° C. to pellet the impurities with the PMPD2-ELP in the supernatant. This completed one full cycle of purification. This was repeated twice for a total of three cycles. After the last cycle the purified PMPD2-ELP was dialyzed against water overnight to get rid of salts. The dialyzed PMPD2-ELP was then lyophilized and stored till further use.

The physical properties of the PMPD2-ELPs were characterized using a UV spectrometer and dynamic light scattering. The biological activity of the PMPD2-ELP was evaluated using neutrophil elastase colorimetric drug discovery kit (Enzo), western blots and proliferation assays of A431 cell line and human skin fibroblasts.

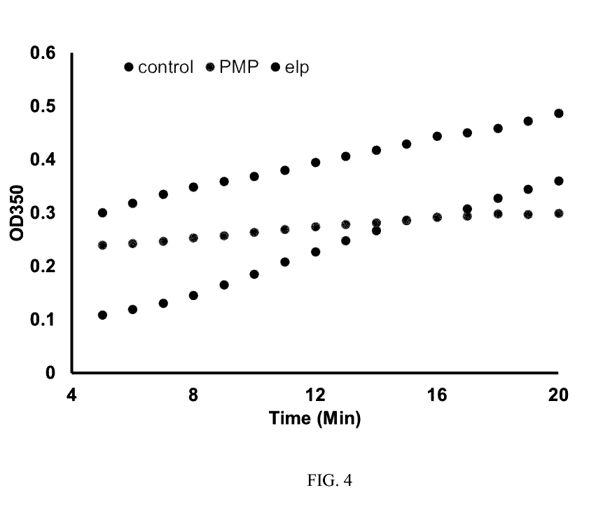

As shown with reference to FIG. 4, elastase activity assay showed that PMP-D2 ELP fusion protein successfully inhibits elastase activity. Spectrophotometer was used to detect the elastase activity every minute starting at 5 min.

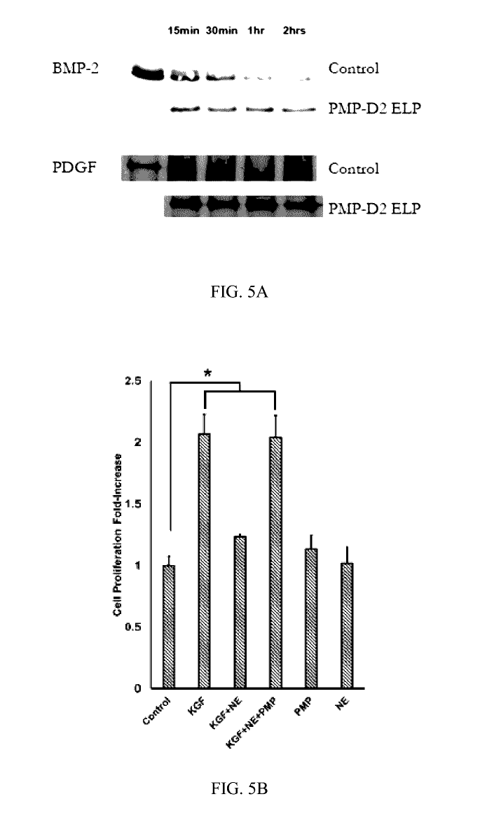

As shown with reference to FIGS. 5A and 5B, PMP-D2 ELP fusion retains the growth factors' bioactivity. In this exemplary embodiment, BMP-2 and PDGF were incubated with elastase, with or without the presence of PMP-D2 ELP, from 15 min up to 2 hours.

As shown with reference to FIG. 5A, a western blot was performed to detect the degradation process of the growth factors by elastase. For BMP-2 without PMP-D2 ELP presence, after 1 hr, more than 50% has been degraded, while with PMP-D2 ELP, BMP-2 activity was retained almost 100%. After 15 min, elastase degraded more than 90% of PDGF, and PMP-D2 preserved 100% of PDGF after 2 hours of incubation.

A431 cells were used to test KGF bioactivity under elastase degradation. KGF was premixed with elastase with or without the presence of PMP-D2 ELP. After half an hour, the mix was put on cells. As shown with reference to FIG. 5B, KGF by itself induced proliferation by 2 fold, and elastase with KGF completely reversed the induced proliferation, while PMP-D2 mix with KGF+NE was able to recover the proliferative activity of KGF on A431 cells.

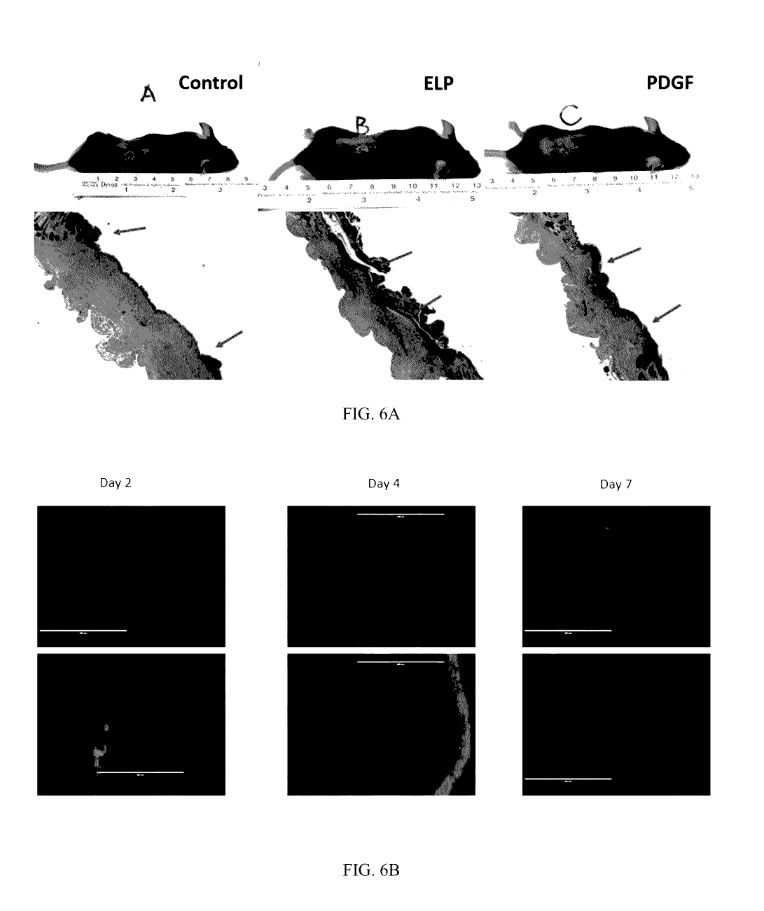

In a preliminary study of ELP in vivo, a diabetic mouse model (B6.BKS-Leprdb) was used to examine the effect of ELP and PDGF on wound healing. Mice were treated with either ELP or PDGF on Day 0 in a fibrin hydrogel, and skin tissues were harvested on Day 14. As shown with reference to FIG. 6A, ELP and PDGF both showed promotion to re-epithelialization when compared to the control.

Diabetic mice were treated with ELP conjugated with biotin to track the degradation process of ELP in vivo. Tissues were stained with Hoechst, and avidin rhodamine was used to label ELP, as illustrated with reference to FIG. 6B.

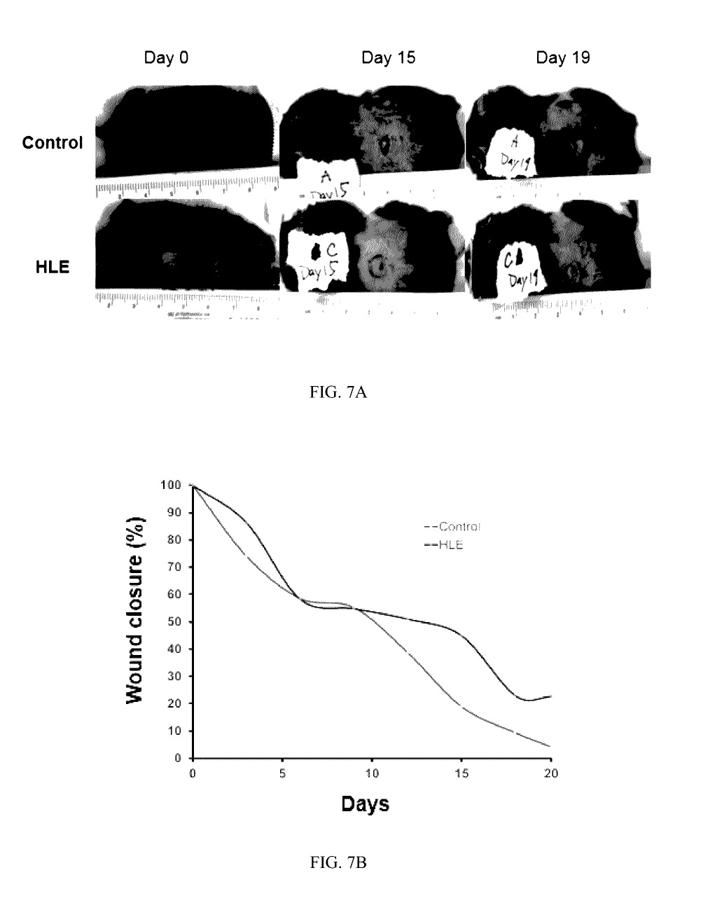

Since elastase level is an important marker for chronic wounds, the method of the present invention creates a novel mouse chronic wound model by adding the serine protease elastase to a full thickness wound. As illustrated with reference to FIGS. 7A and 5B, the data suggests that elastase significantly delayed the wound closure. FIG. 7A illustrates the delay in the wound closure of the mice and FIG. 7B is a graphical illustration of the healing results in the presence of elastase. This novel mouse chronic wound model be used for further study of PMP-D2 ELP in vivo.

The present invention shows that PMP-D2 ELP fusion protein can be successfully expressed and purified and that PMP-D2 ELP fusion protein was not only able to retain its own bioactivity of inhibiting elastase, but also preserve bioactivity of different growth factors that are important to chronic wound healing.

### CONCLUSION

The inventors have shown that a nanoparticle composition comprised of two fusion peptides, a first comprising an elastase resistant peptide conjugated to an ELP and a second comprising a growth factor or other functional peptide conjugated to an ELP, which self-assemble into nanoparticles based on the transition temperature of the ELPs, can be used to successfully treat chronic wounds inexpensively and without repeated administration while still ensuring the bioactivity and stability of the growth factors or functional peptides. While exemplary embodiments are illustrated herein, the invention contemplates the use of any elastase inhibitor, growth factors or functional peptides shown to be efficacious in wound healing and capable of being conjugated to the ELPs.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A nanoparticle composition for chronic wound healing comprising:
a first fusion peptide comprising an elastase resistant peptide bound to a polypeptide backbone;
a second fusion peptide comprising a cargo peptide bound to a polypeptide backbone; and
a pharmaceutically acceptable carrier;
wherein the first fusion peptide and the second fusion peptide self-assemble into a heterogeneous nanoparticle in response to adjustment of transition temperature of the polypeptide backbone.

2. The composition of claim 1, wherein the polypeptide backbone is an elastin-like peptide (ELP).

3. The composition of claim 1, wherein the elastase resistant peptide is a PMP-D2 variant.

4. The composition of claim 3, wherein the PMP-D2 variant is R29L.

5. The composition of claim 3, wherein the PMP-D2 variant is R29L/K30M.

6. The composition of claim 1, wherein the cargo peptide is selected from the group consisting at least one growth factor and functional peptide.

7. The composition of claim 6, wherein the at least one growth factor is selected from the group consisting of epidermal growth factor (EGF); keratinocyte growth factor (KGF); transforming growth factors (TGF); vascular endothelial growth factor (VEGF) including BMP-2; platelet-derived growth factor (PDGF), fibroblast growth factor (FGF); interleukins (IL); colony-stimulating factors (CSF) and combinations thereof.

8. The composition of claim 6, wherein the at least one functional peptide is cathelicidin (LL37).

9. A method of treating chronic wounds in a patient comprising:
administering a nanoparticle composition for chronic wound healing to the patient comprising:
a first fusion peptide comprising an elastase resistant peptide bound to a first elastin-like peptide (ELP);
a second fusion peptide comprising a cargo peptide bound to a second ELP; and
a pharmaceutically acceptable carrier;
wherein the first and second fusion peptides form nanoparticles;
wherein the elastase resistant peptide and the growth factor retain their bioactivity.

10. The method of claim 9, wherein the elastase resistant peptide is a PMP-D2 variant.

11. The method of claim 10, wherein the PMP-D2 variant is R29L.

12. The method of claim 10, wherein the PMP-D2 variant is R29L/K30M.

13. The method of claim 9, wherein the cargo peptide is selected from the group consisting of at least one growth factor and functional peptide.

14. The method of claim 13, wherein the at least one growth factor is selected from the group consisting of epidermal growth factor (EGF); keratinocyte growth factor (KGF); transforming growth factors (TGF); vascular endothelial growth factor (VEGF) including BMP-2; platelet-derived growth factor (PDGF); fibroblast growth factor (FGF); interleukins (IL); colony-stimulating factors (CSF) and combinations thereof.

15. The method of claim 13, wherein the at least one functional peptide is cathelicidin (LL37).
